(19) United States
(12) Patent Application Publication
(10) Pub. No.: US 2015/0164310 A1
(43) Pub. Date: Jun. 18, 2015

(54) INFRARED ILLUMINATED AIRWAY MANAGEMENT DEVICES AND KITS AND METHODS FOR USING THE SAME

(71) Applicant: The Henry M. Jackson Foundation for the Advancement of Military Medicine, Inc., Bethesda, MD (US)

(72) Inventor: Eric Franklin Holt, Bethesda, MD (US)

(21) Appl. No.: 14/414,392

(22) PCT Filed: Jul. 12, 2013
(86) PCT No.: PCT/US2013/050317

§ 371 Date: Jan. 12, 2015

Related U.S. Application Data
(60) Provisional application No. 61/671,379, filed on Jul. 13, 2012.

(51) Int. Cl. A61B 1/06; A61B 1/267; A61B 19/00; A61M 16/04; A61B 1/07

(52) U.S. Cl. CPC A61B 1/063; A61M 16/0488; A61M 16/0447; A61M 16/0486; A61M 16/0463; A61M 16/0461; A61B 1/0684; A61B 1/07; A61B 1/0646; A61B 19/5202; A61B 1/267; A61M 16/0472; A61M 2205/587; A61B 2019/5204; A61M 2205/0216

(57) ABSTRACT

Infrared illuminated airway management devices having an infrared lighting element that can be observed with night vision and/or thermal vision devices and airway management kits including such devices. Endotracheal intubation systems containing a tube introducer having an infrared lighting element. Methods of preparing an open airway by activating an infrared lighting element and inserting at least the distal end of an airway management device into the lumen of an airway, where at least the distal end of the device is illuminated by infrared radiation, as anatomical structures and/or the distal end of the AMD are observed with an infrared detection device. Cricothytoray devices having a retractable cutting edge and an optional infrared lighting element.

### CROSS-REFERENCE TO RELATED APPLICATIONS

This application claims the benefit of, and relies on the filing date of U.S. provisional patent application No. 61/671,379, filed 13 Jul. 2012, the entire disclosure of which is incorporated herein by reference.

### GOVERNMENT INTEREST

This invention was made in part with U.S. Government support. The U.S. Government has certain rights in this invention.

### BACKGROUND

In combat, when a person is injured, one of the first concerns of a battlefield medic is to assure that a wounded person's airway is open and unobstructed. Indeed, airway obstruction is one of the top causes of preventable combat-related death. Trauma and injuries to the face and neck increase the chance of distortion or destruction of a casualty's upper airway anatomy. If a patient's airway is blocked or obstructed, the airway is, if possible, cleared and/or a tube is inserted into the pharynx, larynx, and/or trachea to establish an open airway. The inserted tube permits air to bypass any obstructions and reach the lungs and can prevent the airway from collapsing (e.g., due to a loss of consciousness or additional injury to the wounded person).

An endotracheal intubation is a medical procedure in which a tube is placed into the windpipe (trachea) to administer oxygen, medication, or anesthesia. Landmarks, such as the vocal cords, are used to differentiate the trachea from the esophagus, where the trachea lays on top of the esophagus if the patient is in a supine position. An oxygen tube is inserted into the trachea to provide an open airway. Such oxygen tubes are flexible or not rigid; thus, a somewhat rigid stylet is often used to provide rigidity to the tube while it is being inserted and to provide curvature to the oxygen tube when needed. Once the tube is inserted, the stylet can be disengaged from the oxygen tube and the oxygen tube can be connected to a device to supply oxygen to the patient.

Even in the most convenient settings, it can be a challenge to intubate patients. The airway can become obstructed by fluid, blood, or the patient's own tissues such as the tongue or dislodged teeth. Arthritis involving the cervical spine in the upper neck or restricted mouth opening capability such as is present with individuals with temporomandibular joint (TMJ) dysfunction can make intubation more difficult. Obese individuals can pose an added challenge due to the extra tissue surrounding the airway which often requires skillful manipulation of the airway during intubation. Small children, likewise, have short necks and small jaws, providing a small workspace and making it difficult to locate the vocal cords.

Facial trauma also provides challenges for intubation. Often the anatomy of the person has changed due to the very trauma causing breathing distress, making it more difficult to locate and open an airway. Large overbites also pose a problem as teeth obstruct light, making it difficult for the physician or emergency personnel to view the pharynx and larynx. In addition, a patient's oral cavity may be filled with fluid which also inhibits correct positioning of an airway tube. Emergency personnel or physicians may attempt several times to intubate a patient. Each attempt can add to the trauma suffered by the patient, as the mouth becomes bruised and sore from the various attempts.

When it is difficult to locate the vocal cords of the trachea, often the esophagus is intubated by accident, which can cause the patient to regurgitate stomach contents that can flow into the lungs. This can lead to infection and exacerbate underlying trauma. Thus, devices which facilitate lighting of the oral cavity, the pharynx, larynx, epiglottis and trachea are useful for securing an airway.

Laryngoscopes and stylets having visible light sources to illuminate the oral cavity are available. The light source can help a medical practitioner in being able to see into the relatively dark airway passage of an injured person, so as to make a comprehensive assessment of the person's condition and take the necessary actions to secure the airway. One problem associated with such laryngoscopes and stylets having visible light sources is that during combat, triage and initial stabilization of an injured person is often performed right on the battlefield. Noise and light discipline on the battlefield can necessitate the restriction of visible light use, making direct visualization of an airway (direct laryngoscopy) all but impossible. Furthermore, the additional equipment required for indirect laryngoscopy can be too heavy or impractical to carry in a combat environment. Training and maintaining proficiency for the unique equipment used for indirect visualization of the airway can also be an issue. When a visible light source is on, the emitted visible light can bleed out of the equipment and be detected by the enemy, who can then target the injured person and the medic. However, as the need to secure the airway of a fallen person can be critical and often cannot wait even the few minutes that might be required to transport the injured to a relatively safe rearward position, battlefield medics accept the risk and use visible light when establishing a clear airway even in forward positions under light discipline conditions.

Illuminated laryngoscopes and stylets known in the art use light in the visible or ultraviolet spectrum. There are times where visible light in a combat environment would degrade the operational capability of a unit or team by not only drawing unwanted attention to the casualty or medical provider, but also by interfering with on-going tasks (driving, shooting, flying) being performed by others in the vicinity using night vision and/or thermal vision equipment

Accordingly, there is a need for illuminated airway management devices that are easy to use and not unduly cumbersome and that can be used under low-light conditions without visible light.

### SUMMARY

Certain embodiments are drawn to infrared illuminated airway management devices comprising: an airway management device (AMD), and an infrared (IR) lighting element. The AMD can be an intubating stylet, a bougie, an endotracheal tube, a double lumen airway, an oropharyngeal airway, a nasopharyngeal airway, a laryngeal mask airway, a suction device, a a retrograde intubation guide, or a Magill forceps. The IR lighting element can be removably attached to the AMD or the IR lighting element can be an integral component of the AMD. The IR lighting element can have a thermal signature.

Some embodiments are drawn to airway management kits comprising at least one infrared illuminated airway management device comprising: an airway management device (AMD), and an infrared (IR) lighting element.

Certain embodiments are drawn to an endotracheal intubation system for performing an endotracheal intubation comprising: a tube introducer having an infrared (IR) lighting element, and an endotracheal tube or a double lumen airway. The tube introducer can be an intubating stylet or a bougie.

Some embodiments are drawn to methods of preparing an open airway or an endotracheal conduit through which to administer drugs and/or oxygen comprising: activating an infrared (IR) lighting element, inserting at least a distal end of an airway management device (AMD) into a pharyngeal lumen and/or a tracheal lumen of a subject in need thereof (at least the distal end of the AMD is illuminated by infrared radiation emitted by or transmitted from the activated IR lighting element), and observing anatomical structures of the subject and/or the distal end of the AMD with an infrared detection device. At least the distal end of the AMD is inserted into the pharyngeal lumen ardor the tracheal lumen of the subject in some embodiments.

Certain embodiments are drawn to devices for performing a tracheostomy or cricothyrotomy comprising a retractable cutting edge and, optionally, an infrared (IR) lighting element.

### BRIEF DESCRIPTION OF THE FIGURES

FIGS. 1a-1i illustrate cricothyrotomy devices of certain embodiments.

FIGS. 2a-2o illustrate stylets of certain embodiments.

FIGS. 3a-3h illustrate a design for cricothyrotomy devices of some embodiments.

FIGS. 4a-4d illustrate the positioning of anatomical structures and cricothyrotomy device structures during steps of a cricothyrotomy as used in some embodiments.

FIGS. 5a-5i illustrate stylets with different light/radiation sources and different types of switches, as in certain embodiments.

FIGS. 6a and 6b illustrate the positioning of structures of a cricothyrotomy device during steps of a cricothyrotomy as used in some embodiments.

FIG. 7 is a photograph of a stylet and bougie set/kit of some embodiments. Specific elements of the set are indicated as 7a-7g.

FIG. 8 is a photograph of a cutting edge and bougie of certain embodiments.

FIG. 9 is a photograph of a bougie sledded through a cutting edge as in some embodiments.

FIG. 10 is a photograph of fiberoptics as sources of infrared radiation (a) and visible light (b), as in certain embodiments.

### DETAILED DESCRIPTION

The term “airway management device” (AMD) refers to a medical device used in preparing and/or maintaining an open airway or an endotracheal conduit through which drugs and/or oxygen can be administered. Examples of airway management devices include intubating stylets, bougies, endotracheal tubes, double lumen airways (such as, combitubes, esophageal obturator airways, esophageal gastric tube airways, pharyngeal-tracheal lumen airways, among others), oropharyngeal airways, nasopharyngeal airways, laryngeal mask airways, suction devices (i.e,, aspirators), retrograde intubation guides (i.e., retrograde intubation wires), and Magill forceps.

AMDs for maintaining an open airway can comprise at least one lumen, such as, endotracheal tubes, double lumen airways (such as, combitubes, esophageal obturator airways, esophageal gastric tube airways, pharyngeal-tracheal lumen airways, among others), oropharyngeal airways, nasopharyngeal airways, and laryngeal mask airways, among others known in the art. AMDs for preparing an open airway can include devices for guiding an airway device having a lumen into at least a portion of the natural airway and/or for clearing the natural airway. Such guiding devices can include intubating stylets, bougies, retrograde intubation guides, and Magill forceps. Retrograde intubation guides and suction devices, among other devices known in the art, can be used to clear the airway in preparing an open airway. The term “AMD” or “airway management device” does not encompass a laryngoscope, tracheostomy device, or a cricothyrotomy device.

The term “infrared lighting element” or “IR lighting element” refers to a device providing electromagnetic radiation that can be observed with night vision and/or thermal vision devices, but that is imperceptible or substantially imperceptible to the naked human eye. The electromagnetic radiation emitted by/transmitted from the IR lighting element can fall substantially within the wavelength range of about 600 nm to about 15 μm (which includes infrared radiation overlaps a part of the spectrum of visible light). In some embodiments, the IR lighting element illuminates at least a portion of an AMD with infrared radiation at a wavelength between about 600 nm and about 1000 nm, between about 3 μm and about 5 μm, or between about 7 μm and about 15 μm. The infrared radiation emitted by/transmitted from the IR lighting element can have peak wavelength of about 730 nm, about 830 nm, about 920 nm, or about 940 nm in some embodiments. When the IR lighting element emits/transmits electromagnetic radiation at a wavelength between about 7 μm and about 15 μm it can have a thermal signature,

The IR lighting element can comprise one or more (i.e., an array of) infrared light emitting diodes (IR LEDs) in embodiments. In some embodiments, the IR lighting element can comprise an infrared transmission filtered visible light source. For example, a standard visible light source (such as, an incandescent lamp or fiberoptic visible light source, among others) can be covered with an infrared transmission filter that is designed to pass at least a portion of the visible light source's infrared radiation and block some or all of the visible light component. In certain embodiments, an infrared laser diode can be used as the IR lighting element. An infrared chemiluminescent lighting element can be employed as the IR lighting element in some embodiments. In certain embodiments, the IR lighting element can be sufficiently powerful to permit transluminal (transtracheal) illumination, when viewed with night vision and/or thermal vision devices. In some embodiments, the IR lighting element can he powered by a battery, a chemical reaction, a magnet (Faraday) or mechanical-power generating device, or an external power supply.

Infrared radiation, which falls between the wavelengths of about 750 μm and about 1000 μm in the electromagnetic spectrum, has been variously divided into different categories in the art. IR radiation at a wavelength of about 0.7 μm (700 nm) to about 1.4 μm (1400 nm) is referred to as the near infrared. Night vision devices (such as night vision goggles (NVGs)) can detect radiation at one or more wavelengths (such as at a peak wavelength of about 730 nm, about 830 nm, or about 920 nm, among others) within the near infrared and overlapping slightly into the visible spectrum from about 600 am to about 700 am, or about 600 nm to about 750 nm. Certain night vision devices can detect low level visible light falling within the visible spectrum from about 600 nm to about 750 nm in addition to infrared radiation.

Night vision devices (NVDs) are optical instruments that allow images to be produced in levels of light approaching total darkness. Some night vision devices amplify existing levels of available light/radiation and convert the near infrared radiation to a wavelength visible to humans. Certain NVDs can collect small amounts of light/radiation, including the lower portion of the infrared spectrum (wavelengths between about 750 nm and about 1400 nm) and optionally, parts of the visible spectrum between the wavelengths of about 600 nm to about 750 nm, present in the environment that may be imperceptible to the observer's eyes, and amplify them to the point that an image can be observed.

Thermal imaging devices operate by capturing the upper portion of the infrared spectrum (at wavelengths between about 7 μm and about 15 μm), which is emitted as heat by objects (a thermal signature), instead of simply capturing reflected radiation in the near infrared. Hotter objects, such as warm human/animal bodies will emit more of this radiation than cooler objects like trees or buildings. Enhanced night vision devices (ENVDs) employ image-intensifying and thermal-imaging technologies, together or individually. ENVDs can be used to detect near infrared, visible light and/or thermal signature.

A specific example of an enhanced night vision device is the AN/PSQ-20 Enhanced Night-Vision Goggle (ENVG), among others. The AN/PSQ-20 ENVG can provide improved target detection. The AN/PSQ-20 ENVG is a monocular passive night vision device developed for the United States military by ITT EXELIS. The AN/PSQ-20 ENVG combines image-intensifying and thermal-imaging technologies, enabling vision in conditions with very little light. The two technologies can be used simultaneously or individually, when using the AN/PSQ-20 ENVG. Prior to the development of the AN/PSQ-20 ENVG image intensifier and thermal imaging could only be used separately. The AN/PSQ-20 ENVG is classified as a third-generation passive night vision device and can provide vision through thermal imaging even in situations where there is insufficient ambient light for the effective use of image intensifiers, thus eliminating the need for active night vision. The AN/PSQ-20 ENVG can be used to see through obscurants such as smoke and fog. The combined technologies allow better target identification and recognition, thereby improving a user's mobility and situational awareness.

Examples of night vision, thermal vision, and enhanced night vision devices include cameras, goggles, and scopes, among others, with night vision and/or thermal vision capabilities. The devices can comprise image intensification, thermal signature detection and/or active illumination elements.

Certain embodiments are drawn to infrared illuminated airway management devices comprising: an airway management device (AMD), and an infrared (IR) lighting element. An AMD can comprise one or multiple lumens/tubes for (a) delivering air, oxygen, volatile anesthetics or other gases, (b) suctioning debris, (c) suctioning or delivering fluids or medicines, or (d) inserting a malleable stylet, a fiberoptic scope or fiber, a therapeutic instrument or tool, or a medically or tactically necessary therapy or material. The AMD can include one or more ON/OFF selector-type switches. Such switches can be guarded, toggled, or timed, and can have multiple settings for using one, none, or multiple combinations of illumination (i.e., visible light and infrared radiation). In some embodiments, an infrared illuminated airway management device comprising an infrared lighting element can further comprise a visible light source. The infrared lighting clement can have a thermal signature in some embodiments. In certain embodiments, an infrared illuminated airway management device can comprise a source of visible light, near infrared radiation, infrared radiation, and/or light/radiation compatible with thermal imaging devices.

In sonic embodiments the AMD can comprise a cutting device, which can be disposable, retractable, guarded, monopolar, bipolar, electric, or manual for assisting in acquiring access to a patient's or casualty's airway. In embodiments, infrared illuminated AMDs can be of variable diameters, thicknesses, malleability characteristics, and lengths and the IR illuminated AMDs can have variable sizes for a primary or secondary lumen. The characteristics of an IR illuminated AMD used in certain embodiments can depend on the function (acting as a guide, or oxygenation and ventilation) of the AMD. In embodiments, an AMD can be pre-shaped or be malleable (capable of being shaped as needed) to assist with intubation or cannulation of an airway. In certain embodiments, the shape of an endotracheal tube or double lumen airway can be adjusted by using a stylet or bougie. In some embodiments, the AMD (such as, an endotracheal tube or a double lumen airway) can have an inflatable cuff with a lumen or tube connected to the cuff for use in inflating or deflating the cuff in order to prevent unwanted flow of air or fluids around the exterior of the device, while positioned in a patient's natural airway.

In embodiments, the AMD can be an intubating stylet, a bougie, an endotracheal tube, a double lumen airway, an oropharyngeal airway, a nasopharyngeal airway, a laryngeal mask airway, a suction device, a retrograde intubation guide, or a Magill forceps. In some embodiments, the AMD can be an intubating stylet or an endotracheal tube.

The IR lighting element can comprise (a) an infrared light emitting diode (IR LED), (b) an near-infrared light emitting diode, (c) an infrared transmission filtered visible light source, (d) an infrared laser diode, (e) a fiberoptic source, or (f) an infrared chemiluminescent lighting element in embodiments. In certain embodiments, the IR lighting element can comprise an infrared transmission filtered visible light source. In some embodiments, the IR lighting element can be removably attached to the AMD. In other embodiments, the IR lighting element can be an integral component of the AMD (that cannot be removed from the AMD). The infrared lighting element may project light/radiation in one or multiple directions. The IR lighting element can have a thermal signature in certain embodiments. An IR illuminated airway management device may include and be capable of using an on-board or external power/voltage source.

In embodiments, the AMD can have a proximal end and a distal end, and during an airway management procedure (such as endotracheal intubation, among others) the distal end can be introduced into the pharyngeal lumen or the tracheal lumen. In some embodiments, the infrared (IR) lighting element can illuminate at least the distal end of the AMD during an airway management procedure.

Certain embodiments are drawn to airway management kits comprising at least one infrared illuminated airway management device comprising: an airway management device (AMD), and an infrared (IR) lighting element. In some embodiments, the kit can further comprise a laryngoscope, optionally also comprising an infrared lighting element. In some embodiments, the airway management kit can comprise a plurality of infrared transmission filters, wherein each infrared transmission filter transmits radiation of a different range of wavelengths and the IR lighting element used with the AMD comprises a visible light source and an infrared transmission filter selected from the plurality of infrared transmission filters in the kit. In some embodiments the kit comprises an infrared illuminated AMD comprising an IR lighting element comprising a visible light source and a transmission filter that transmits electromagnetic radiation at a wavelength between about 600 nm and about 1000 nm, while blocking other wavelengths of visible light.

Some embodiments are drawn to endotracheal intubation systems for performing an endotracheal intubation comprising: a tube introducer, and an endotracheal tube or a double lumen airway. In embodiments the tube introducer has an infrared (IR) lighting element and the tube introducer can be an intubating stylet or a bougie.

Certain embodiments are drawn to methods of preparing an open airway or an endotracheal conduit through which to administer drugs and/or oxygen. The methods can comprise: activating an infrared (IR) lighting element, inserting at least a distal end of an airway management device (AMD) into a pharyngeal lumen and/or a tracheal lumen of a subject in need thereof (at least the distal end of the AMD is illuminated by infrared radiation emitted by/transmitted from the activated IR lighting element), and observing anatomical structures of the subject and/or the distal end of the AMD with an infrared detection device, as at least the distal end of the AMD is inserted into the pharyngeal lumen and/or the tracheal lumen of the subject. In embodiments the infrared detection device can be a night vision device and/or a thermal vision device, such as night vision goggles or enhanced night vision goggles, among others. In some aspects, the radiation emitted by/transmitted from the activated IR lighting element can have a wavelength between about 600 nm and about 15 μm. In some methods, the AMD can be an intubating stylet, a bougie, an endotracheal tube, a double lumen airway, a laryngeal mask airway, or a retrograde intubation guide. In certain embodiments, the IR lighting element can be attached to the AMD and the method can further comprise attaching the IR lighting element to the AMD before or after it is activated to emit/transmit light/radiation. In some embodiments, the IR lighting element can be an integral component of the AMD. In certain methods, the anatomical structures used as landmarks when securing an airway can be observed directly. In some embodiments, the infrared radiation emitted by/transmitted from the IR lighting element can be observed transluminally.

Embodiments can solve problems associated with securing a patient's or a casualty's airway in nonclinical, pre-hospital, tactical, and unconventional environments, especially with poor lighting or where visible light is contraindicated. Embodiments can provide airway management devices for use in securing a casualty's or a patient's airway using radiation at wavelengths in the visible, infrared (optionally at wavelengths providing a thermal signature), or near-infrared spectrum. Infrared illuminated airway management devices can be used in standard clinical settings and/or in unconventional, austere or tactical environments.

Laryngoscopy is a medical procedure used to obtain a view of the vocal cords and the glottis and certain embodiments can comprise laryngoscopy. Laryngoscopy can be either direct or indirect. Direct laryngoscopy is done with an unaided direct line of sight and can be performed by inserting a laryngoscope into the right side of a patient's mouth, moving the tongue to the left in order to sight the epiglottis, which is then displaced anteriorly to provide an unobstructed view of the glottic opening of the trachea. Indirect laryngoscopy accomplishes the same objective, but with the aid of additional visual equipment such as fiberoptic bronchoscopes or stylets, video laryngoscopes, or optically-enhanced laryngoscopes that incorporate mirrors or prisms. Either type of laryngoscopy (direct or indirect) known in the art can make use of a typical (unlighted) or lighted stylet. Certain embodiments comprise laryngoscopy (direct or indirect) with a stylet having an infrared lighting element.

A type of stylet known in the art that can be used in embodiments herein is a malleable metal plastic-coated rod slightly longer in length than an endotracheal tube (ETT) into which it is inserted. The stylet can be pre-formed, and in some embodiments, the stylet can have a small curled pull end (for grasping and removing the style: from the ETT once intubation is accomplished) and a slight curve along its entire length in order to facilitate intubation. The stylet can be used to provide an ETT with a certain measure of rigidity during insertion and its form can assist in navigating around the tongue, saliva, and soft tissue of the upper airway. While it is often possible to intubate a person without the use of a styleted ETT, it is common practice to have at least one ETT pre-styleted for difficult airway intubations in emergency airway kits. Certain embodiments comprise a stylet illuminated with infrared radiation.

One type of stylet known in the art is a stylet with a visible light source at its distal end. If an ETT is loaded over the stylet, the distal visible light source can illuminate the airway to assist with direct laryngoscopy. Another technique, called trans-tracheal illumination, involves advancing a lighted stylet into an airway until a faint glow can be observed through the skin of the neck, with subsequent passage of the ETT over the stylet to complete intubation. In embodiments a stylet can be illuminated with infrared radiation that is substantially not visible or invisible to the naked human eye during laryngoscopy/intubation. Infrared radiation can be used for direct laryngoscopy or trans-tracheal illumination while observing with a night vision and/or thermal vision device in embodiments.

In the art, ETTs can have their own visible light sources. As discussed above, endotracheal intubation is a medical procedure that can be used to “secure an airway”, allowing ventilation and oxygenation of a patient or casualty. An intubation procedure can involve inserting an ETT into the mouth of a patient, past the tongue, through the glottic opening and past the vocal cords. A breathing circuit or bag valve mask can be connected to the end of the ETT protruding from the patient's airway to supply or remove air, oxygen, inhaled volatile anesthetics, or other gases or materials. Either direct or indirect laryngoscopy can be used to perform one of two main types of endotracheal intubation, orotracheal and nasotracheal intubation. In the former, the patient is intubated by introducing an endotracheal tube (ETT) through the mouth, while the latter involves insertion of an ETT through the nose. Certain embodiments comprise an ETT illuminated with infrared radiation. Some embodiments comprise orotracheal or nasotracheal intubation using an infrared lighting element.

Cricothyrotomy and tracheotomy are additional methods of securing a patient's airway. Both procedures involve cannulating the trachea with an incision made through the skin and into the tracheal lumen. These procedures can be performed when orotracheal and nasotracheal intubation attempts have been unsuccessful or are contraindicated. A cricothyrotomy, although quicker to perform than a tracheotomy, is a temporizing measure until a more definitive airway can be obtained. Certain embodiments comprise performing a cricothyrotomy or tracheotomy using a device illuminated with infrared radiation.

Another procedure that can be used to secure an individual's airway is retrograde intubation. In retrograde intubation, the cricothyroid membrane is punctured with a large-bore needle (i.e., 16 or 14-guage) and a guide (such as, a central venous guide wire, among others) is inserted cephalad through the needle, up the larynx, and out the mouth. An ETT (or other airway tube) can then be advanced into the trachea over the guide. Other devices, such as a fiberoptic bronchoscope can be used in place of a central venous guide wire to obtain the same result.

Challenges of retrograde intubation include requirement for an incision or puncture through the skin and into the trachea, and increased risk of bleeding, damage to adjacent structures (such as, nerves and arteries), improper incision location, infection, and possible introduction of unintended foreign bodies into the airway. The guide used in retrograde intubation can also provide challenges. In some cases, a guide (such as, a central venous guide wire) employed can be too flexible to function as a proper ETT guide and it can be too thin and therefore too difficult to grasp and manipulate, especially in a pre-hospital environment, where manual dexterity is often degraded due to fatigue, stress, gloves, or other environmental conditions. Certain embodiments can comprise performing retrograde intubation using a guide illuminated with infrared radiation.

Night vision and thermal vision devices/equipment (such as ENVGs, among others) can be standard issue for members of the military, law-enforcement, and other agencies. Night vision and thermal vision devices can be used in low-light and/or tactical environments. Individuals can drive, fly, shoot, read, and perform other tasks using existing night vision and/or thermal vision technology. In embodiments, infrared illuminated airway management devices are compatible with night vision and/or thermal vision equipment. Certain embodiments can permit a battlefield medic or first responder to assess and secure the airway of a wounded person, while minimizing the risk of being spotted due to visible light bleed or interfering with others in the vicinity performing functions while using night vision or thermal vision technology.

Certain embodiments permit a medic or first responder, aided by night vision and/or thermal vision equipment to secure a subject's airway in darkness or near-darkness using methods comprising direct/indirect laryngoscopy, transluminal (transtracheal) illumination, retrograde intubation, cricothyrotomy, or tracheotomy. Using certain embodiments, light discipline can be maintained, while performing potentially life-saving medical care.

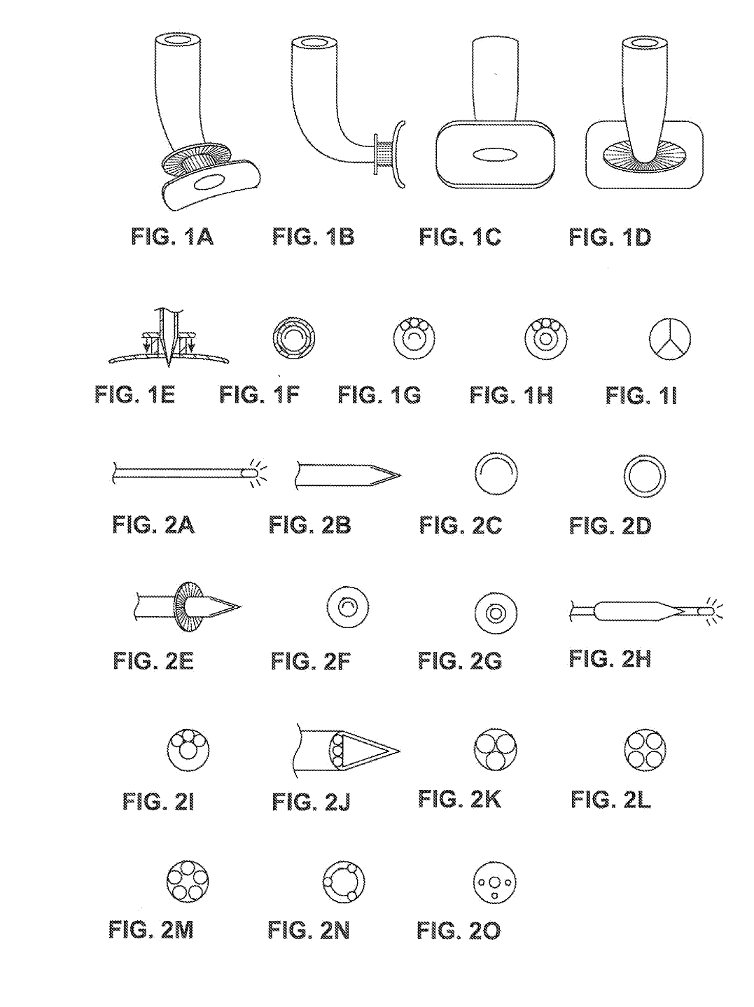

Certain embodiments can be better understood by reference to the Drawings. FIG. 1 illustrates a cricothyrotomy device of certain embodiments. FIG. 1a is an oblique view of the cricothyrotomy device. In FIG. 1a the mesh-like outer layer has a spring that permits retraction of the awl-shaped sharp cutting edge, after it is used to cut an incision into a patient's neck. FIG. 1b is a side view, FIG. 1c is a front view and FIG. 1d is a back view of the cricothyrotomy device. FIG. 1e is a longitudinal-sectional view of part of FIG. 1a, and FIG. 1f is a cross-sectional view of part of FIG. 1a. FIG. 1f illustrates the outer layer spring and the curved sharp awl-shaped cutting edge (represented by an open half circle). FIG. 1g is cross-sectional view of part of FIG. 1a, illustrating the half circle shaped cutting edge and three LED (light emitting diode) lumens: one for an LED capable of producing radiation having a thermal signature (Th) (e.g., between about 7 μm to about 15 μm), another for an LED capable of producing visible light, and still another for an LED capable of producing infrared radiation at both wavelengths with and without a thermal signature (long-wave infrared (e.g., between about 7 μm to about 15 μm) and near infrared (IR/NIR)(e.g., between about 0.7 μm and 1.4 μm)). Once the cutting edge is retracted during cricothyrotomy, each of the light/radiation sources can help a practitioner (i.e., medic) visualize a person's airway. FIG. 1h is a cross-sectional view of the cutting edge with three LED lumens as shown in FIG. 1g with an awl-shaped cutting edge. FIG. 1i is a design for a lumen with three compartments for LEDs (thermal signature providing LED, visible light providing LED, and IR/NIR providing LED). The IR/NR LED can produce light/radiation at wavelengths that can be detected by both night vision devices and thermal imaging devices. FIG. 1a-α depicts an element of the cricothyrotomy device that is placed flush against the neck. The element 1a-α can be curved to better tit to the neck and aid in properly positioning the cricothyrotomy device when incising the cricothyroid. The spring cutting mechanism as shown in more detail in FIG. 1e can prevent the user from making too deep an incision during a cricothyrotomy.

FIG. 2 illustrates a stylet of some embodiments. FIG. 2a illustrates a stylet with an LED. FIG. 2b depicts a stylet with a cutting edge and without a stopper. FIG. 2c is a cross-sectional view of FIG. 2b with a half circle shaped cutting edge. FIG. 2d is a cross-sectional view of FIG. 2b with an awl-shaped cutting edge. FIG. 2e depicts a stylet with a cutting edge and a stopper to allow retraction of the cutting edge. FIG. 2f is a cross-sectional view of FIG. 2e with a half circle shaped cutting edge. FIG. 2g is a cross-sectional view of FIG. 2e with an awl-shaped cutting edge. FIG. 2h illustrates a stylet having a rough surface handle that can aid in grasping the stylet properly, as when hands grasping the stylet are wet or cold. FIGS. 2i-2o illustrate aspects of a stylet/boogie/ETT of certain embodiments with various lumen configurations for accommodating different light/radiation sources, gases and/or drugs. Each circle represents a lumen including: 1) lumens for different types of lighting elements (IR, NIR, visible light, or chemiluminescence light/radiation sources), 2) lumens capable of acting as conduits for gases oxygen), water or fluids with electrolytes/drugs, and/or 3) lumens that contain an source for producing a thermal signature.

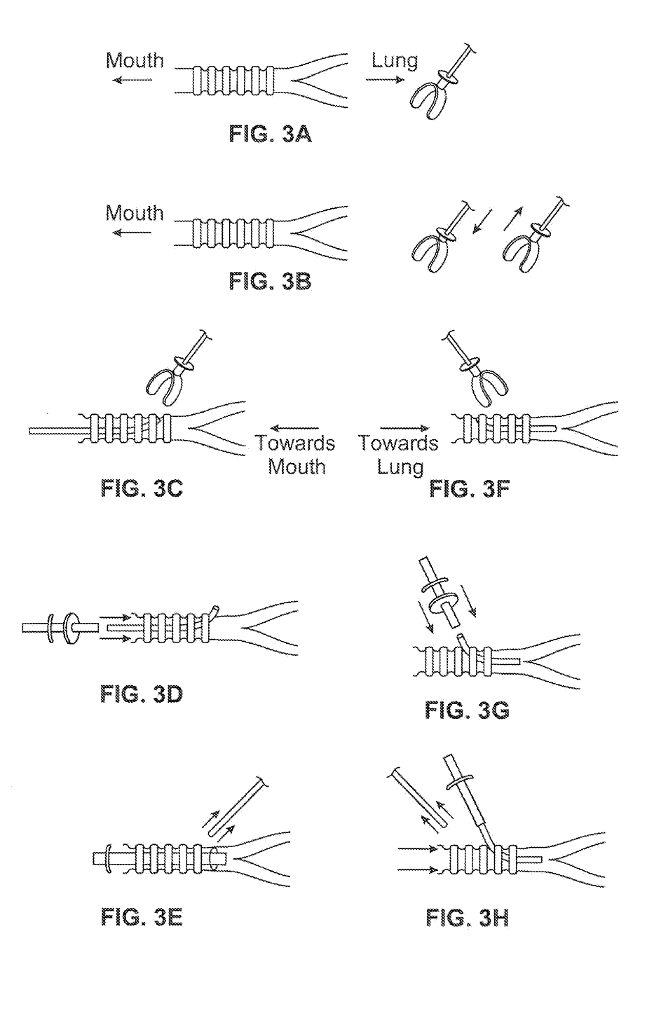

FIGS. 3a, 3b, 3c and 3f illustrate a design for a cricothyrotomy device (see also FIGS. 1a-1e) having an clement (e.g., an anatomical guide) that is curved when brought into contact with a person's neck, that aids in locating the cricothyroid membrane/cartilage and properly positioning of the cricothyrotomy device and that reduces the likelihood of making too deep an incision during a cricothyrotomy. The cricothyrotomy device illustrated in FIG. 3 can avoid a cutting edge (such as a scalpel) from entering unnecessarily deep into the cricothyroid during a cricothyrotomy. The cricothyrotomy device of FIG. 3 can have an anatomical guide that is curved to fit the neck and that can make it easier for practitioners to locate the cricothyroid cartilage (see FIG. 1a-α). FIGS. 3a-3e depict the airway passage including the trachea and the path to the lungs. FIGS. 3c-3e illustrate an intubation tube entering through the mouth and exiting through the incision at the cricothyroid cartilage. In contrast. FIGS. 3f-3h illustrate an intubation tube entering from the incision at the cricothyroid and threading through the trachea toward the lungs.

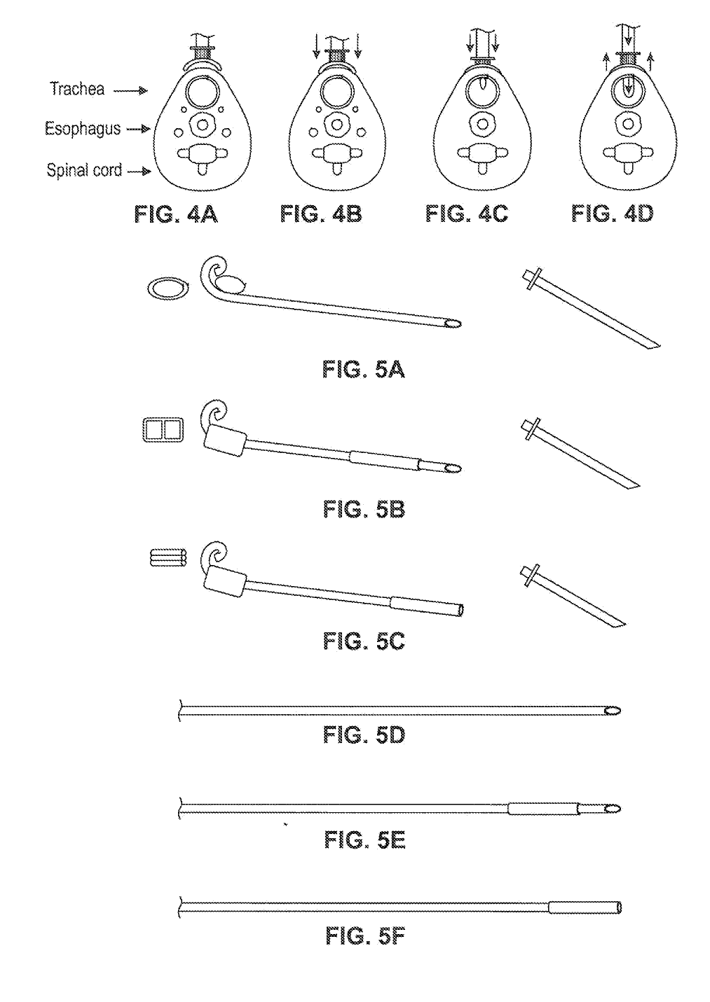

FIG. 4 illustrates the positioning of anatomical structures and cricothyrotomy device structures during steps of a cricothyrotomy using the cricothyrotomy device illustrated in more detail in FIGS. 3a, 3b, 3c and 3f and FIGS. 1a-1e. FIGS. 4a and 4b illustrate the structures as the anatomical guide of the cricothyrotomy device is used to position the device on the neck. FIG. 4c illustrates the structures as the cutting mechanism in the device is used to puncture the cricothyroid membrane and cartilage. FIG. 4d illustrates the structures as the cutting mechanism is retracted after making an incision in the neck. Although FIGS. 1a-1e, 3a, 3h, 3c, 3f, and 4a-4d illustrate a design for a cricothyrotomy device, elements depicted in these figures can also be employed in certain embodiments drawn to tracheostomy devices.

Certain embodiments are drawn to devices for performing a tracheostomy or cricothyrotomy comprising a retractable cutting edge. In some embodiments, a tracheostomy device or cricothyrotomy device comprises an infrared (IR) lighting element in addition to the retractable cutting edge. The tracheostomy/cricothyrotomy device can comprise an infrared (IR) lighting element having a thermal signature in some embodiments. In certain embodiments, an IR lighting element can be an integral component of the tracheostomy/cricothyrotomy device. In some embodiments, the tracheostomy or cricothyrotomy device can comprise at least two lumens for housing a visible light source and an infrared (IR) lighting element or at least three lumens (one for an LED capable of producing radiation having a thermal signature (Th) (e.g., between about 7 μm to about 15 μm), another for an LED capable of producing visible light, and still another for an LED capable of producing infrared radiation at both wavelengths with and without a thermal signature (long-wave infrared (e.g., between about 7 μm to about 15 μm) and near infrared (IR/NIR) (e.g., between about 0.7 μm and 1.4 μm)). The light sources used with the tracheostomy/cricothyrotomy device can be positioned for use in locating the incision site, for insertion of a tube/instrument into the incision, and for visualization of the airway.

The retractable cutting edge can be a cutting edge known in the art. The cutting edge can be a beveled half-circle, full circle, or crescent, among others known in the art. The length of the retractable cutting edge can be varied depending on the retraction mechanism and the incision site, so that the incision is made to the proper depth for establishing an airway while minimizing unnecessary injury to the tissue. In certain embodiments, the tracheostomy or cricothyrotomy device can have depth gauge markings for use by a technician in avoiding incising too deeply when employing the retractable cutting edge. The depth gauge markings can be in metric or English measures and in sonic embodiments, can be visible with use of an IR lighting element,

In some embodiments, the retractable cutting edge can be push-spring deployed with automatic recovery into a safety position once thumb pressure or tweezer-type pincer pressure is removed. In certain embodiments, the retractable cutting edge can have a curved shape on the lower part of the lumen so it does not cover the area being visualized during the tissue incision/penetration. The retractable cutting edge can comprise metal, plastic, composite, or ceramic, among other materials known in the art, and the retractable cutting edge can be radio-opaque or radiolucent, in certain, embodiments.

in some embodiments, the tracheostomy or cricothyrotomy device can comprise a retraction mechanism comprising a spring to retract the retractable cutting edge. The retraction mechanism can be spring loaded, guarded, covered, and/or disposable, in certain embodiments. In some embodiments, the tracheostomy or cricothyrotomy device can comprise two or more lumens. Optional lumens (in addition for those for accommodating a visible light source and an IR lighting clement) can include lumens permitting suction, visualization, introduction of medication, and introduction of instrumentation.

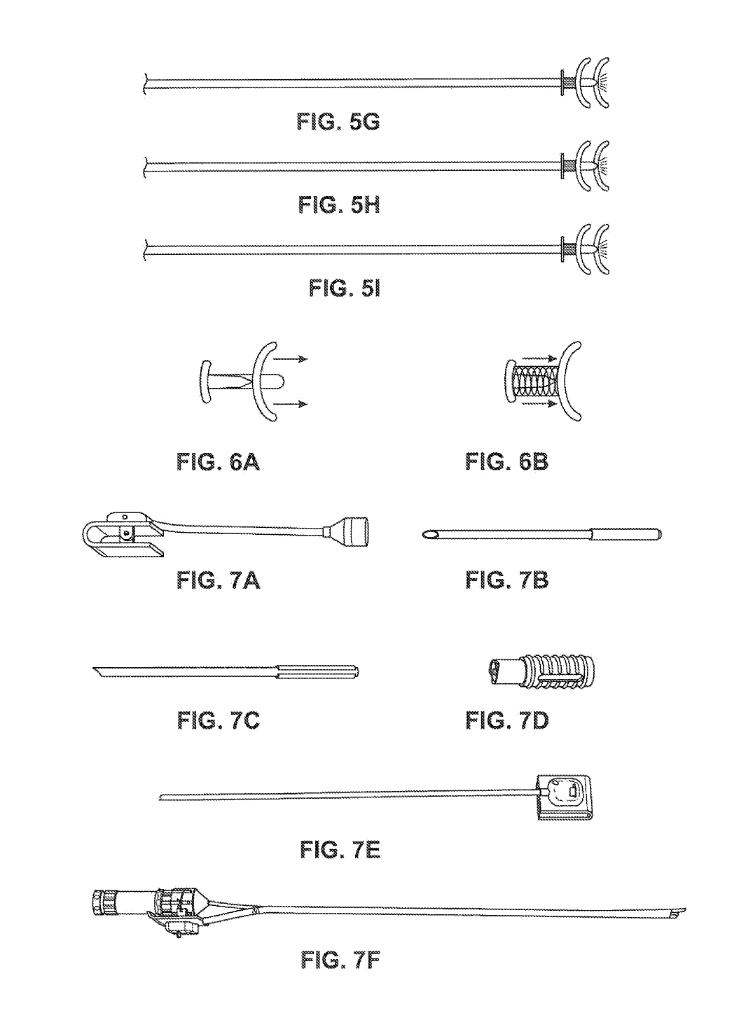

One of the main concerns with cricothyrotomy (and tracheostomy) is locating the correct spot on the neck to perform the procedure. The trachea is easily palpated and has a convex surface that stands out against the lateral musculature and other structures in a young healthy adult. In some embodiments, the tracheostomy or cricothyrotomy device can comprise an anatomical guide to aid in proper placement of the device for incision by the retractable cutting edge. Anatomical guides in certain embodiments can have a curved backstop that can not only assist in locating and ‘fixing’ the trachea in place, but can also provide a platform from which to gauge the depth that the retractable cutting edge should enter the tissue. A curved trachea contouring backstop can also be used to secure the device with a strap around the back of the neck to hold it in place. In certain embodiments, a strap may be attached to the backstop/anatomical guide for securing the device. FIG. 5 depicts stylets with different light/radiation sources and different types of switches, as in some embodiments. FIG. 5a depicts a stylet that can be provided with one or more different light/radiation sources ((1) visible light source, (2) I/NIR (long-wave infrared and near infrared) source, or (3) chemiluminescent source) and a pressure switch. FIG. 5b illustrates a stylet that can be provided with one or more different light/radiation sources and a click type switch. FIG. 5c depicts a stylet that can be provided with one or more different light/radiation sources and a twist type switch. FIGS. 5d-5f illustrate stylets that can be provided with one or more different light/radiation sources that are used to enter the airway passage from mouth. FIGS. 5g-5i illustrate stylets that can be provided with one or more different light/radiation sources that are used to enter the airway passage from the cricothyroid.

FIGS. 6a and 6b are similar to similar to FIGS. 4c and 4d, except that the cross-section of a human neck is not shown in FIG. 6. FIG. 6a depicts the cutting edge of the cricothyrotomy device as it is used for incision. FIG. 6b depicts the cutting edge as it is in the retracted position. FIGS. 6c-6f depict other aspects of the cricothyrotomy device in certain embodiments.

The following Examples further define and describe embodiments herein. Unless otherwise indicated, all parts and percentages are by weight.

### EXAMPLES

Production of Infrared Illuminated Airway Management Devices and Cricrothyrotomy Devices

1. Malleable aluminum tubes were used to build a) a stylet, b) a bougie, and c) a cutting edge for a cricothyrotomy device. Diameters of the tubes ranged from about 1/16 of an inch to about 5/32 of an inch.

2. 20-30 gauge solid copper and insulated wire (as can be found in telephone/computer cables) was inserted through the walls of the aluminum tubes described in 1.

3. Near infrared (N(IR)) LEDs were connected to the wire in 2. The wavelength of N(IR) LED was 940 nm and the physical measurements of the LED were a diameter of 0.200 in., LED head 0.340 in., and lead length 1.0 in.

4. Low voltage switches and small button batteries (3×1.3 (1.5) V) were connected to the wire in 2 that was connected to the N(IR) LED in 3.

5. A very small direct current was used to connect the battery, wire and LED.

Photographs of infrared Illuminated Airway Management Devices Produced and Kit Containing the Same

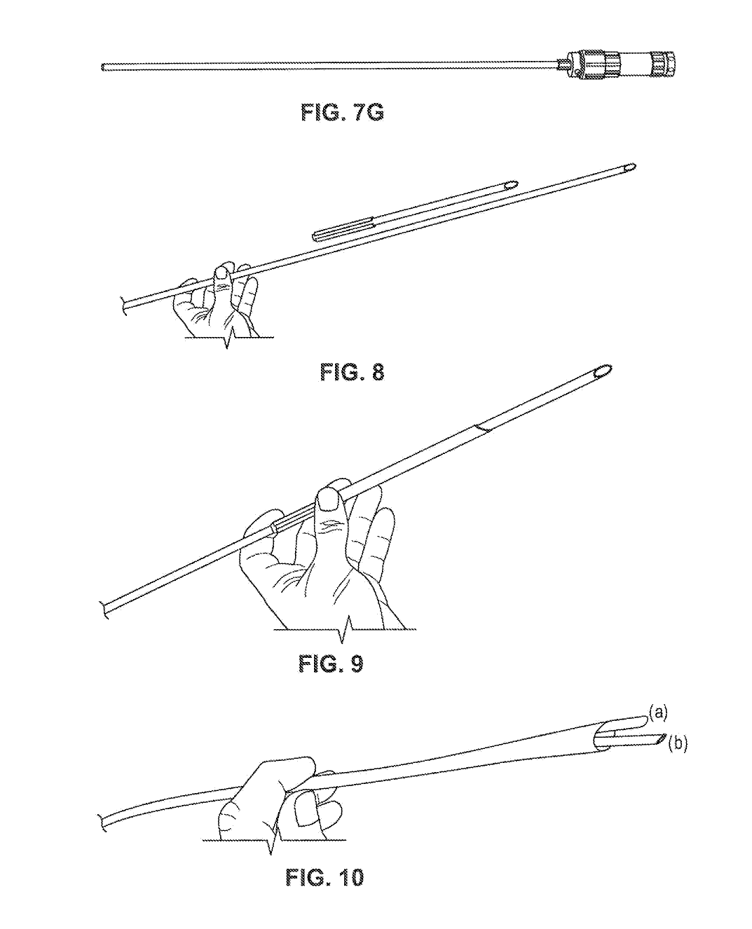

FIGS. 7-10 are photographs of airway management devices of embodiments. FIG. 7 is a photograph of a stylet and bougie set. 7a is a flashlight for providing infrared illumination. 7b and 7c are cutting edges. 7d is a light/radiation source for the flashlight 7a. 7e is a stylet with a visible light source. 7f is a stylet with infrared and visible light sources that can be detected with night vision devices (a near infrared (NIR) source that can emit radiation having a thermal signature could be employed). 7g is a bougie with an IR source. The cutting edges 7b and 7c can be inserted into stylet 7e, stylet 7f, or bougie 7g, for use during securing/managing an airway. (See FIGS. 8 and 9.)

FIG. 8 is a photograph of cutting edge 7b and bougie 7g lying next to each other. FIG. 9 is a photograph of bougie 7g, sledded through cutting edge 7b. FIG. 10 is a photograph of an IR lighting clement with a fiberoptic source for infrared radiation 10a capable of being detected with a night vision device and a fiberoptic source for visible light 10b.

While the present teachings have been illustrated with respect to one or more implementations, alterations and/or modifications can be made to the disclosed embodiments without departing from the spirit and scope of the appended claims. In addition, while a particular feature of the present teachings may have been disclosed with respect to only one of several implementations, such feature may be combined with one or more other features of the other implementations as may be desired and advantageous for any given or particular function.

To the extent that the terms “containing,” “including,” “includes,” “having,” “has,” “with,” or variants thereof are used in either the detailed description and the claims, such terms are intended to be inclusive in a manner similar to the term “comprising.” As used herein, the term “one or more of” with respect to a listing of items such as, for example, A and B, means A alone, B alone, or A and B. The term “at least one of” is used to mean one or more of the listed items can be selected.

Notwithstanding that the numerical ranges and parameters setting forth the broad scope of the present teachings are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 5, In certain cases, the numerical values as stated for the parameter can take on negative values. In this case, the example value of range stated as “less than 10” can assume values as defined earlier plus negative values, e.g., −1, −1,2, −1.89, −2, −2.5, −3, −10, −20, and −30, etc.

1. An infrared illuminated airway management device comprising:
an airway management device (AMD), and
an infrared (IR) lighting element.

2. The infrared illuminated airway management device of claim 1, wherein the infrared (IR) lighting element has a thermal signature.

3. The infrared illuminated airway management device of claim 1, wherein the AMD is an intubating stylet, a bougie, an endotracheal tube, a double lumen airway, an oropharyngeal airway, a nasopharyngeal airway, a laryngeal mask airway, a suction device, a retrograde intubation guide, or a Magill forceps.

4. The infrared illuminated airway management device of claim 1, wherein the IR lighting element comprises (a) an infrared light emitting diode (IR LED), (b) a near-infrared light emitting diode, (c) an infrared transmission filtered visible light source, (d) a infrared laser diode, (e) a fiberoptic source, or (f) an infrared chemiluminescent lighting element.

5. The infrared illuminated airway management device of claim 1, wherein the IR lighting element is removably attached to the AMD.

6. The infrared illuminated airway management device of claim 1, wherein the IR lighting element is an integral component of the AMD.

7. The infrared illuminated airway management device of claims 1, wherein the AMD has a proximal end and a distal end and during an airway management procedure the distal end is introduced into the pharyngeal lumen or the tracheal lumen.

8. The infrared illuminated airway management device of claim 7, wherein the infrared (IR) lighting element illuminates at least the distal end of the AMD during an airway management procedure.

9. An airway management kit comprising at least one infrared illuminated airway management device according to claim 1.

10. The airway management kit of claim 9, further comprising a laryngoscope.

11. The airway management kit of claim 10, wherein the laryngoscope also comprises an infrared lighting element.

12. The airway management kit of claim 9, wherein the kit comprises a plurality of infrared transmission filters, wherein each infrared transmission filter transmits infrared radiation of a different range of wavelengths and wherein the IR lighting element comprises a visible light source and an infrared transmission filter selected from the plurality of infrared transmission filters.

13. The airway management kit of claim 12, wherein the selected transmission filter of the IR lighting element transmits infrared radiation at a wavelength between about 600 nm and about 1000 nm.

14. An endotracheal intubation system for performing an endotracheal intubation comprising:
a tube introducer, and
an endotracheal tube or a double lumen airway,
wherein the tube introducer has an infrared (IR) lighting element and the tube introducer is an intubating stylet or a bougie.

15. A method of preparing an open airway or an endotracheal conduit through which to administer drugs and/or oxygen comprising:
activating an infrared (IR) lighting element,
inserting at least a distal end of an airway management device (AMD) into a pharyngeal lumen and/or a tracheal lumen of a subject in need thereof, wherein at least the distal end of the AMD is illuminated by infrared radiation emitted by or transmitted from the activated IR lighting element, and
observing anatomical structures of the subject and/or the distal end of the AMD with an infrared detection device, as at least the distal end of the AMD is inserted into the pharyngeal lumen and/or the tracheal lumen of the subject.

16. The method of claim 15, wherein the infrared detection device is a night vision device, a thermal vision device, or a combination thereof.

17. The method of claim 15, wherein the infrared radiation emitted by or transmitted from the activated IR lighting element has a wavelength between about 600 nm and about 15 μm.

18. The method of claim 15, wherein the AMD is an intubating stylet, a bougie, an endotracheal tube, a double lumen airway, a laryngeal mask airway, or a retrograde intubation guide.

19. The method of claim 15, wherein the IR lighting element is attached to the AMD and the method further comprises attaching the IR lighting element to the AMD.

20. The method of claims 15, wherein the IR lighting element is an integral component of the AMD.

21. The method of claim 15, wherein the anatomical structures are observed directly.

22. The method of claim 15, wherein the infrared radiation is observed transluminally.

23. A device for performing a tracheostomy or cricothyrotomy comprising a retractable cutting edge.

24. The device of claim 23, further comprising an infrared (IR) lighting element.

25. The device of claim 24, wherein the infrared (IR) lighting element has a thermal signature.

26. The device of claim 24, wherein the IR lighting element is an integral component of the device.

27. The device of claim 23, comprising a retraction mechanism comprising a spring to retract the retractable cutting edge.

28. The device of claim 23, comprising at least two lumens for housing a visible light source and an infrared (IR) lighting element.

29. The device of claim 23, further comprising an anatomical guide.
